(19) United States
(12) Patent Application Publication
(10) Pub. No.: US 2020/0040273 A1
(43) Pub. Date: Feb. 6, 2020

(54) WOOD PROCESSING METHOD

(71) Applicant: GLOMMEN TECHNOLOGY AS, Elverum (NO)

(72) Inventor: Arne Johannes GRONN, Hernes (NO)

(73) Assignee: GLOMMEN TECHNOLOGY AS, Elverum (NO)

(21) Appl. No.: 16/594,849

(22) Filed: Oct. 7, 2019

Related U.S. Application Data
(63) Continuation-in-part of application No. 15/316,347, filed on Dec. 6, 2016, now Pat. No. 10,435,640.

(30) Foreign Application Priority Data
Jun. 6, 2014 (GB) 1410101.8

(51) Int. Cl. C10L 5/44; C10L 5/36; C10L 9/08; D21C 1/02; D21C 11/00; C12P 19/14; C08B 37/00

(52) U.S. Cl. CPC C10L 5/44; C10L 5/363; C10L 9/08; C08B 37/0003; D21C 11/0007; C12P 19/14; D21C 1/02

(57) ABSTRACT

The invention provides a method for generating a solid wood-based material and a hemicellulose-derived material from a wood raw material. The method includes treating the wood raw material under aqueous conditions at elevated temperature and pressure to generate a hemicellulose-containing fluid component and a solid component; separating the fluid component from the solid component; processing at least a part of the solid component into a solid wood-based material; and processing the liquid component into a hemicellulose-derived material. The invention also provides for a wood-derived fuel with a low ash content.

### CROSS REFERENCE TO RELATED APPLICATIONS

This application is a continuation-in-part of U.S. patent application Ser. No. 15/316,347, filed Dec. 6, 2016, which is a U.S. National Phase Application of PCT/EP2015/062624, filed Jun. 5, 2015, which claims the benefit of GB 1410101.8 filed on Jun. 6, 2014. The entire contents of these applications are incorporated by reference in this application.

### TECHNICAL FIELD

The present invention relates to the generation of fuels and other valuable materials from a wood or other biomass raw material.

### BACKGROUND

Wood Pellets

Wood fuel has always been important. Today advanced wood fuel in the form of pellets is an alternative to fossil fuels. A broad range of furnaces can be modified to use wood pellets instead of coal. For a significant part of such furnaces, the wood fuel is burnt as powder. The powder is obtained by milling wood pellets, but can also be made just by milling dry wood.

Most wood pellets are so-called “white pellets”, which is made from wood that has been dried to about 10% moisture, grinded, and compressed in pellets mills to pellets of typically 6 or 8 mm diameter, lengths typically from 5-20 mm. These pellets return to the form as wood powder if exposed to water, which is a disadvantage. There is great interest in finding a way to produce hydrophobic wood pellets.

Torrified pellets is one solution for hydrophobic wood pellets. Another solution is pellets made from wood which has been steam exploded. Such pellets are also to a large degree hydrophobic, but not totally.

As the quantity of wood being used as raw material for wood pellets increases, the costs of raw materials may rise. While sawdust used to be the main raw material for wood pellets, today ordinary cellulose chips and pulpwood are being used as raw material for wood pellets. This requires that the use of the wood be done in a way to get the most value out of it.

The present invention represents, in the first instance, a way to get increased value for the wood. This is done by separating the hemicellulose from those parts of the wood going to be pelletized and using the hemicellulose for other products. Furthermore, the resulting material may have additional properties which improve its suitability for uses such as fuels.

The present invention is also concerned in particular with the treatment of other, non-wood, lignocellulosic biomass for use as fuel. Such biomass can advantageously be processed to be used in place of wood. It would evidently be an advantage to use waste biomass, such as materials left over from agricultural processes, in place of wood as fuel since wood potentially has a higher value in other uses. Separation of biomass into high-value products has evident advantages.

One of the most important criteria for typical combustion of such fuels is the ash deformation temperature. For wood fuels that is usually at an acceptable level when treated with standard processes. However, for other lignocellulosic materials, like straw, bagasse, and similar, the deformation temperature for the ash is so low that it can create problems with the combustion equipment. Without being bound by theory, this is believed to be caused by sintering of the ash at combustion temperatures. As a result of the low ash deformation temperature of fuels derived from non-wood materials, the combustion equipment requires such frequent cleaning. This means that fuels with low ash melting temperatures cannot, in practice, be used on a commercial scale. Evidently it would be an advantage to provide a method for converting biomass into fuel with a high ash deformation temperature. It would be a further advantage if this process could utilise a variety of biomass materials as starting materials and particularly if the process could utilise non-wood biomass since this may be waste material or material of lower value.

Another problem with fuels deriving from lignocellulosic materials other than wood is that the chlorine content can often be too high, which leads to problems with the equipment.

The present inventors have surprisingly found a process which enables the formation of biomass-derived fuel which addresses at least one, preferably all, of the issues listed above and/or provides at least one of the advantages indicated herein.

### SUMMARY

Prior art concerning making wood pellets from wood that has been steam exploded is described in BRUSLETTO (WO/2006/006863A1), GRØNN (US20110302832 A1), and HARRIS (US20110296748 A1). These patents describe various methods for treatment of the wood with steam before making pellets.

Although previous methods are effective in the formation of wood pellets, it would evidently be a considerable advantage to generate additional value from the raw material during the production of wood pellets or other biomass materials. Contrary to previous methods, the present inventors have now established that by appropriate separation and processing procedures, a wood raw material or other biomass material can be separated into high-energy components for the formation of fuel and high-value components for additional uses. The methods of the invention may also provide other advantages, particularly to the fuel material.

In the present invention, hemicellulose is extracted from the wood or other biomass material. Thereafter, the hemicellulose is processed further for uses other than being a component of wood/biomass pellets. The remaining components of the wood or other biomass, mainly consisting of cellulose and lignin, are made to wood pellets, or wood powder fuel, or other products.

In a first aspect, the present invention therefore provides a method for generating a solid, wood-based material (such as a fuel material) and a hemicellulose-derived material from a wood raw material, said method comprising;
i) treating the wood raw material under aqueous conditions at elevated temperature and pressure whereby to generate a hemicellulose-containing fluid component and a solid component;
ii) separating said fluid component from said solid component;
iii) processing at least a part of said solid component into solid, wood-based material (e.g. a fuel); and
iv) processing said liquid component into a hemicellulose-derived material.

In a further aspect, the invention provides a method for generating a solid biomass-derived material and a hemicellulose-derived material from a biomass raw material, said method comprising;
i) treating the biomass raw material under aqueous conditions at elevated temperature and pressure whereby to generate a hemicellulose-containing fluid component and a solid component;
ii) separating said fluid component from said solid component;
iii) processing at least a part of said solid component into a solid biomass-based material;
wherein said biomass is a lignocellulosic material;
wherein said solid biomass-derived material has an ash deformation temperature of at least 1000° C.

In a further aspect, the invention provides a solid biomass-derived material having an ash deformation temperature of at least 1000° C. an ash content of less than 0.25 wt %,
wherein said biomass is a non-wood lignocellulosic material.

In a further aspect, the invention provides a liquid fuel comprising the solid biomass-derived material defined herein and at least one hydrocarbon liquid.

In a further aspect, the invention provides a solid biomass-derived material obtained or obtainable by the method described herein.

The main components of wood/biomass are cellulose, lignin and hemicellulose, of which cellulose is the largest component. The percentage distribution varies with the wood/biomass species. The energy density of these main components is very different. While cellulose has an energy density not far from the average energy density in the wood, the lignin has an energy density per weight unit significantly above that. The hemicellulose has an energy density per weight unit significantly lower than the average for wood. The approximate energy content of lignin is about 27 MJ/kg, for cellulose about 18 MJ/kg and for hemicellulose below 15 MJ/kg. Removal of hemicellulose thus increases the energy density of the remainder.

By separating the (lower energy density) hemicellulose from the rest of the wood/biomass (e.g. before pelletizing), we therefore increase the energy density in the fuel (e.g. fuel pellets or fuel powder) made from the remaining parts of the wood/biomass. If we then can use the hemicellulose for products with better value than as being part of a wood/biomass fuel (or wood/biomass pellets), then we have increased the total value of the wood.

Most of the ash content in the wood/biomass becomes water soluble after the steam treatment. The method of the present invention thus serves to dissolve the water soluble part of the ash, which is then removed from the solids fraction, and thus from the final solid wood-based material product or solid biomass-derived material product. The fuel product therefore has a very low ash content, compared to other wood-based fuels or other biomass-derived fuels. The ash content of the solid wood or solid biomass fraction is observed to be even lower than in heavy oil fuel. It can therefore be used in combustion equipment designed for oil or gas fuels, which generally cannot be used for ordinary wood fuels, and even in combustion equipment without ash handling.

In one embodiment, appropriate to all aspects of the present invention, the solid wood-based material or solid biomass-derived material is a fuel with an ash content of no more that 0.25% by weight. Preferably this solid wood-based material or solid biomass-derived material will have an ash content of no more than 0.15 wt % (which is the maximum amount of ash permitted in heavy fuel oil), more preferably no more than 0.1 wt % and most preferably no more than 0.08%, 0.06%, 0.05%, or 0.04 wt %. Most preferably, the solid wood-based material or solid biomass-derived material will be a fuel (e.g. fuel pellets or fuel powder) with an ash content as indicated, and most preferably no more than 0.3% by weight. Wood derived fuels or solid biomass-derived materials with an ash content below 0.25 wt % are not generally available and thus in a further aspect, the present invention provides a wood fuel (e.g. a wood fuel pellet or a wood fuel powder) or solid biomass-derived material having an ash content as indicated herein.

In a further embodiment, the solid biomass-derived material has an ash deformation temperature of at least 1000° C. (e.g. 1000° C. to 2000° C.), preferably at least 1050° C., preferably at least 1100° C., preferably at least 1200° C., more preferably at least 1300° C., e.g. at least 1400° C. Ash deformation temperature for solid fuel is usually required at 1100° C. Suitable ash deformation temperature ranges include 1000-2000° C., e.g. 1200-1800° C. The ash deformation temperature is herein typically measured using method SIS-CEN/TS15370-1:2007.

In previous efforts, additives for increasing the ash melting temperature, such as mineral agents, e.g. calcium carbonate, lime or limestone are generally added to solid fuels to avoid the problems associated with low ash deformation temperatures. It would evidently be an advantage in terms of cost and/or complexity to avoid the need of additives, particularly mineral agents (e.g. calcium carbonate, lime or limestone).

The process of the present invention is beneficial in that high deformation temperatures can be achieved. This avoids or reduces the need to add such materials. In the present invention, the process is therefore typically carried out in the absence of additives for increasing the ash melting temperature, such as mineral agents, e.g. calcium carbonate, lime or limestone. In a further embodiment, the ash deformation temperature is measured in the absence of such additives for increasing the ash melting temperature.

In a further embodiment, the solid biomass-derived material has a chlorine content of 0.2 wt % or less, preferably 0.1 wt % or less, more preferably 0.08 wt % or less, more preferably 0.07 wt % or less, more preferably 0.06 wt % or less, more preferably 0.05 wt % or less, more preferably 0.03 wt % or less. Suitable chlorine content ranges include 0.005-0.2 wt % or 0.01-0.1 wt %. The percentages are herein typically expressed as dry basis wt %. The chlorine content is herein typically measured using method SS-ENISO16994:2016.

### BRIEF DESCRIPTION OF THE DRAWINGS

FIG. 1 shows a schematic representation of an example method of the present invention.

FIGS. 2A-2D show four micrographs a) to d) each showing particles generated by the method of the present invention. Smallest dimensions of some of the larger particles are shown in microns (μm).

FIG. 3 shows the particle size distribution of wood particles of the present invention as measured by laser scattering using a Malvern Mastersizer 2000 laser scattering instrument.

### DETAILED DESCRIPTION

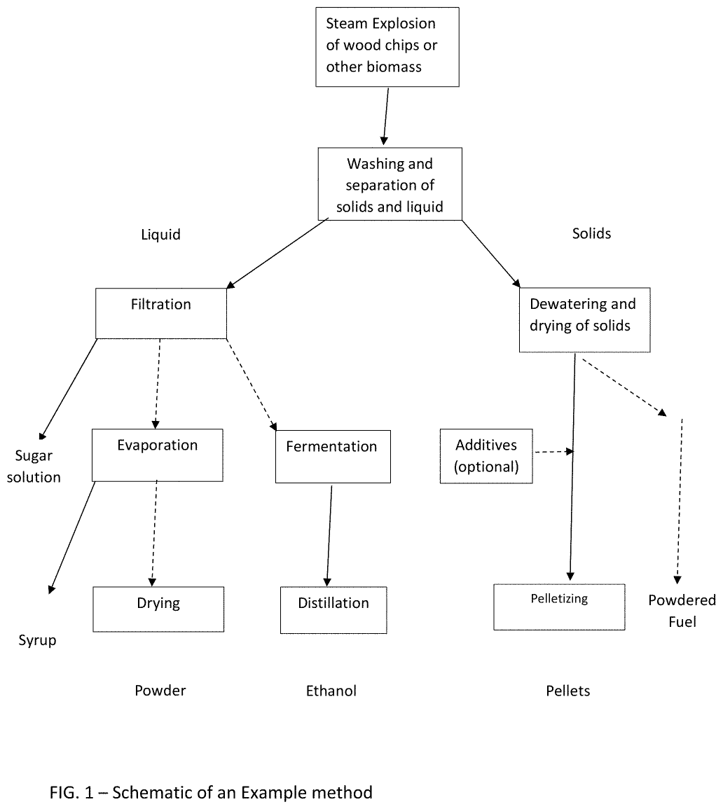

The main elements of one key embodiment of the method according to the present invention are illustrated in the diagram of FIG. 1.

In FIG. 1, it can be seen that the method typically begins with steam treatment (steam explosion) of wood chips. This serves several functions, as discussed herein including helping to liberate the hemicellulose and/or helping to solubilise the ash content. The second step of the Example method then separates the hemicellulose from the cellulose and lignin by washing and phase separation. The liquid phase is then filtered and used to generate a sugar solution, a syrup or a sugar-containing powder or is fermented and optionally distilled to generate ethanol. The solid component is at least partially dried and used to generate solid materials such as fuels. The fuels may be in the form of pellets as illustrated in FIG. 1 or may equally advantageously be in other forms such as a powder (as discussed herein).

Previous methods of separating hemicellulose from wood have been described, for example by RETSINA (U.S. Pat. No. 8,518,672B2, US20130244291A1, US20130309728A1). Retsina does not, however, use the present method or relate to the current advantageous combination of a fuel-generating method and a hemicellulose-product generating method.

### Steam Treatment (Steam Explosion)

The first step is steam treatment (also called steam explosion) of wood and/or other biomass materials. Both terms steam explosion and steam treatment will be used interchangeably in the following text, with the same meaning. The wood may be hardwood or softwood, in the form or woodchips or smaller particles. The (other) biomass raw material can be as described herein. The wood or biomass can have natural moisture, or being more or less dried.

The main parameters for the steam explosion (steam treatment) are:
Temperature 150-230° C. (e.g. 180 to 230° C.)
Temperature reached by injection of steam into a pressure vessel containing wood
Cooking time 120-1200 seconds

The temperature is reached by injecting steam into a pressure vessel containing wood and/or the other biomass raw material. If the steam is saturated, the pressure and temperature will follow a defined path. If the steam is super-heated, then the pressure will be lower at a given temperature than if the steam is saturated.

Suitable temperatures for steam treatment (explosion) include 150-230° C., 190-230° C., 190-220° C., 195-220° C., 195-215° C. Suitable cooking times for steam treatment (explosion) include a time of more than 60 seconds, e.g. more than 120 seconds, e.g. 60-2400 seconds, preferably 60-1200 seconds. Particular preferred combinations are steam treatments at 190-230° C. for a period of 60-1200 seconds. Preferred cooking time for hardwood is 120-720 seconds at temperature in the range of 190-215° C., such as 195-215° C. Preferred cooking time for softwood is 180-600 seconds at temperature in the range of 195-215° C., e.g. 200-212° C.

The pressure release at the end of the steam treatment cycle is done in one or more (e.g. at least two) steps. The pressure may first be reduced by releasing steam to another vessel without blowing out any significant quantity of wood/biomass particles. Thereafter the pressure is released and going to ambient by blowing out the remaining steam and wood/biomass in one blow. Alternatively, the pressure may be released in a single step.

The lower the cooking temperature, then the longer cooking time is needed in order to process the wood/biomass. These process parameters must be adjusted according to which wood/biomass species are being processed. The particle size and moisture content also influence& the optimal parameters.

Optimal parameters are those parameters that lead to the highest yield in extraction of hemicellulose, without reducing the quality for the following steps of the solids and liquid fractions.

In one variant, some of the pressure is reduced by injecting water into the pressure vessel. The processed wood/biomass will then be in the form of a slurry when the vessel is emptied, and the slurry goes to a washing and separation step.

The wood raw material used in the methods of the present invention may comprise hardwood, softwood or a mixture thereof. The material will generally be in the form of pieces, such as chips, dust or other particles. Typical particle sizes will range in largest dimension from around 10 cm to around 1 mm.

By biomass raw material is meant a lignocellulosic material. Such a material could comprise, for example, at least 1 wt % lignin and at least 1 wt % cellulose. The term lignocellulosic material is well known in the art. The biomass typically comprises, for example, agricultural residues (or ‘agroresidues’). Any typical biomass is suitable for treatment according to the present invention, including, but not limited to, straw, bagasse, stover, bamboo stems and/or leaves, fibrous residues from rice and cereal processing, grass, stem, pod or other waste materials from crop plants (e.g. oilseed rape stems or pods, potato, pea or bean stems, or pods of peas or beans) and any mixtures thereof, preferably straw and bagasse, or mixtures thereof. The biomass can include:
virgin biomass (which includes all naturally occurring terrestrial plants such as trees, bushes and grass, preferably non-wood bushes and grass),
waste biomass (which is produced as a low value byproduct of various industrial sectors such as agriculture (corn stover, sugarcane bagasse, straw etc.) and forestry (saw mill and paper mill discards) preferably agriculture), or
energy crops (crops with high yield of lignocellulosic biomass produced to serve as a raw material for production of second generation biofuel—e.g. switch grass and elephant grass)

Preferably, the biomass material is a non-wood lignocellulosic material, i.e. it is a lignocellulosic material which is other than wood, typically other than tree wood. Biomass material may be treated “whole” or may be broken, chopped etc. into pieces for treatment.

### Washing and Separation of Solids and Liquid

The hemicellulose becomes water soluble when being exposed to steam treatment (steam explosion), something that is well known. The second step comprises washing and separation of the solids and liquid fractions. During this, the hemicellulose is extracted from the wood/biomass, and is in the solution. The washing step may also serve to remove at least a part of the ash content which may be rendered soluble by the steam treatment step.

It was surprisingly found that the separation of the solids and liquid results in an increase in the ash deformation temperature for the solids fraction, in particular for biomass raw materials (e.g. non-wood biomass). Components causing low deformation temperature are typically removed from the solid material in the separation step. This is highly beneficial for the processing of biomass materials which typically have low ash deformation temperatures (e.g. non-wood such as straw, bagasse etc.).

The separation also surprisingly results in a reduction in the chlorine content of these biomass materials. Such materials generally have high chlorine contents (e.g. >0.4 wt %) which are unsuitable for typical combustion equipment. The process of the present invention results in suitable fuels derived from biomass materials which would otherwise typically have high chlorine content (e.g. straw, bagasse etc.).

In some variants, after washing, but before separation, enzymes enabling hydrolysis of part of the cellulose is added, and the separation may be delayed by up to 36 hours while hydrolysis takes place. In this variant, parts of the cellulose will be converted to glucose, and become water-soluble.

In some variants, the separation takes place by using for example dewatering screws that bring the moisture level in the solids fraction below 50% moisture on a wet basis.

### Dewatering and Drying of Solids

This step comprises a drying step, for which a broad range of dryer types can be used. This step may also comprise mechanical dewatering, for example by dewatering screw, before the use of a dryer.

### Ash Content

Most of the ash content is rendered soluble by the method of the present invention and is removed by the washing step. The solid component thus has a very low ash content, which may be less than 0.15%, or even less than 0.1, 0.08, 0.07 or 0.05%. Even lower ash contents are achievable as indicated herein.

As a consequence of the low ash content, the solid component is compatible with ash requirements for traditional gas turbines, or the powdered solid component can be mixed with liquid hydrocarbons in liquid fuel burners. The present invention thus additionally provides for a fuel, for example a gaseous or liquid fuel comprising wood particles, or other biomass-derived materials, having a very low ash content as described herein. Such wood particles or biomass-derived materials may be formed or formable by the methods described herein. Such a fuel may be a gaseous fuel in which wood particles or biomass-derived materials such as the solid component described herein are suspended in a fuel gas (e.g. methane), an oxidising gas (e.g. oxygen or air) or an inert gas (e.g., nitrogen). Similarly, such a fuel may be a liquid fuel in which wood particles such as the solid component described herein are suspended in a fuel liquid (e.g. a liquid hydrocarbon or hydrocarbon mixture such as fuel oil).

### Solid Component Fuel

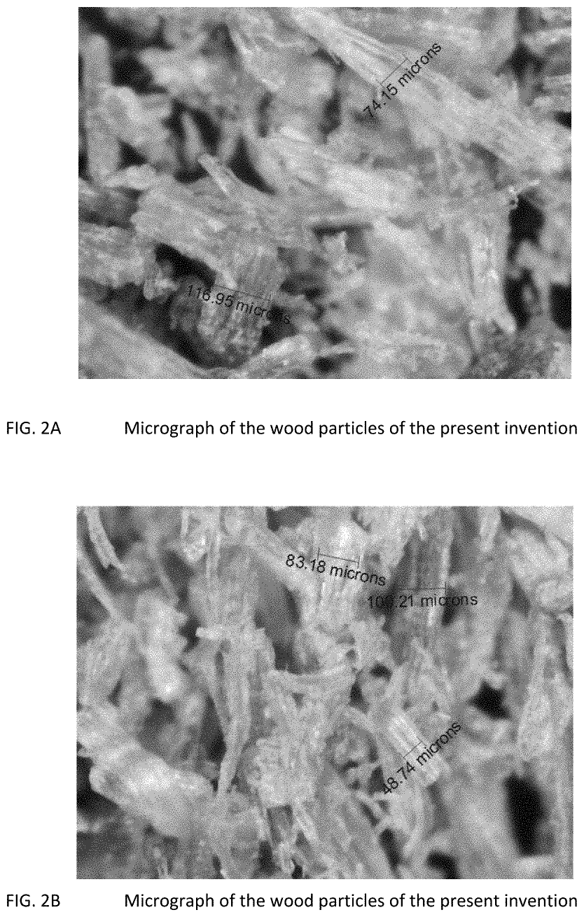

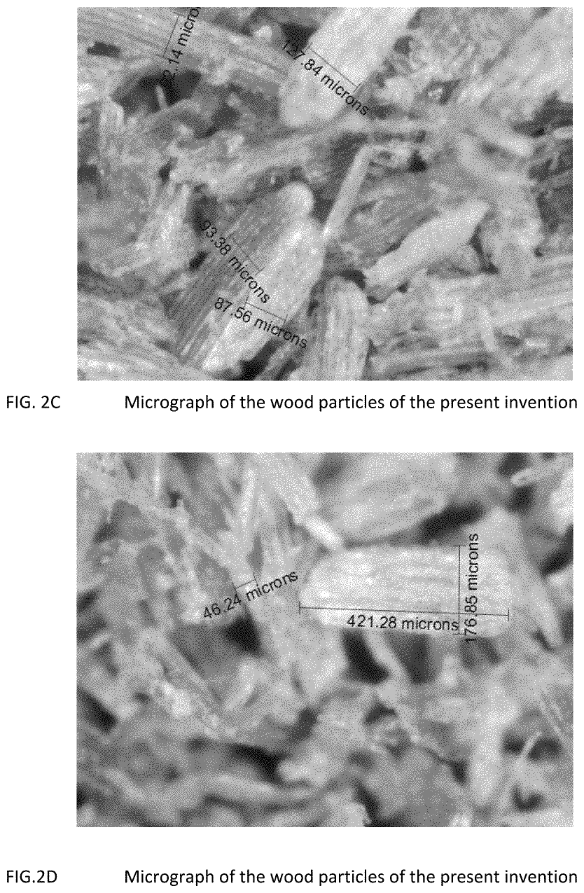

A further advantage of the method of the present invention is that the resulting particles of solid component (also referred to herein as wood particles or biomass particles) may have a very favourable size and/or size distribution. It has been observed that the powder produced through this process has a fine granulometry with the smallest dimension of at least 80% (preferably at least 90%) of the particles being less than 250 μm (e.g. as measured by microscopy). Generally the smallest dimension will be less than 200 μm in 80% or preferably 90% of particles (by number) and most preferably less than 150 μm. The particles are typically asymmetric as a result of the grain in the wood raw material and generally have one longer dimension and two smaller dimensions. Without being bound by theory, the advantageous combustion properties are at least partially attributed to the particles being small in their smallest dimension, as indicated herein, because the combustion front will progress through the smallest dimension. Wood particles or biomass-derived particles of the present invention may thus show immediate and full combustion where powders with larger particles can sometimes show non-burnt particles. This measurement of smallest dimension may be made effectively by microscopy (see FIGS. 2A-2D).

Given this small granulometry, and provided the low ash content described above, The powder form of the solid component fuel can directly be used in gas turbines and/or fuel burners (mixed with liquid fuel) without customisation of the turbine/burner. This provides very valuable flexibility for feeding burners.

FIGS. 2A-2D show micrographs of the typical wood particles formed by the method of the present invention with dimensions illustrated in micrometres (microns). It can be seen that only the larger particles are measured and these generally have a smallest dimension below 250 μm and often still smaller.

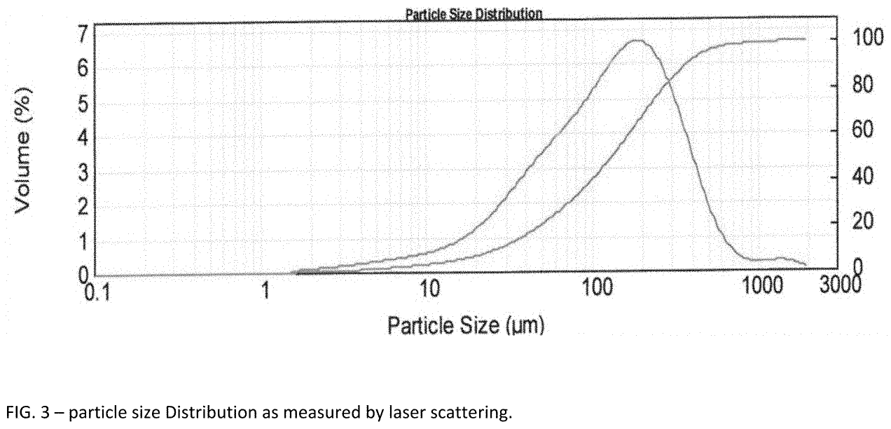

Particle sizes were also measured using a Malvern Mastersizer 2000 laser scattering instrument, a typical result from which is illustrated in FIG. 3. It can be seen that by volume %, around 90% of the sample is less than 400 μm, but this laser scattering instrument does not effectively measure the smallest dimension, which is the most relevant dimension in the present context. Correspondingly, when measured by laser scattering, the largest or average dimension is likely to be more closely represented then the smallest dimension. The larger particles may also be over-represented due to the nature of the instrument and the tendency of fibrous particles to agglomerate.

Thus, in a related embodiment, the wood particles or solid biomass-derived particles of the present invention may be such that at least 60% by volume have a particle size below 250 μm when measured by laser scattering.

### Optional Additives

Optional additives are of different types. One type is substances rich in fat or oil, which will improve energy content, binding and hydrophobic properties of the pellets.

Another type of additive is carbon rich substances that increases the energy content and the fixed carbon in the solids (e.g. pellets). Among such substances are coal and charcoal dust. Pellets made with these additives may be used as reducing agents in the metallurgical industry.

### Pelletizing of Dried Solids

Due to low or none content of hemicellulose, the properties relevant for pelletizing are different from steam exploded wood. Hemicellulose is to some extent a binder if present during pelletizing. To get just as good binding properties for steam exploded wood from which the hemicellulose has been separated, the cooking time during the steam explosion must be long enough, or the temperature in the die during compression to pellets must be higher, or additives rich in fat or oil might be used.

As hemicellulose is water soluble, the absence of hemicellulose increases the hydrophobic properties of pellets.

In an alternative embodiment, the dried solids may be formed into any solid material, such as a construction material for structural and/or decorative uses. Such construction materials will be well known in the art and include beams, sheets, boards, mouldings etc. The formation of such materials may be by well-known techniques and may optionally incorporate a binder such as a resin binder.

### Enzyme Treatment

The solid component or fraction in the methods of the present invention may at any suitable stage be treated in order to cause partial hydrolysis of the cellulose. This may, for example occur after a steam explosion step, or after separation of the solid component from the fluid component. Such hydrolysis will typically be carried out for a period of 1 to 72 hours, particularly 1 to 36 hours and will be followed by a separation step. The solid component from that separation will then be processed into a solid material as described herein and the liquid may be treated separately or may be combined with the hemicellulose-containing fraction and treated with that fraction. Typically the hydrolysed fraction will be processed into similar products as described herein with regard to the hemicellulose fraction, such as sugar solution, syrup, sugar-containing powder and/or fermentation products (e.g. ethanol, methanol, acetic acid etc).

### Filtration

Filtration or separation may be carried out in any number of steps, typically proceeding from most course filtration to most fine filtration. A single separation step may be used but generally at least two separation steps will be needed; a first to remove suspended material and a second (nano- or ultra-filtration) to increase the concentration of dissolved material. The invention can nonetheless be carried out using the first separation step only, e.g. if further processing using nano- or ultra-filtration is not essential. Multiple steps including increasingly fine filtration steps and/or a plurality of ultrafiltration steps may be used depending upon the nature of the fluid component and the final product.

The first separation (e.g. filtration) step is in order to remove fibres and other particles. The first step may involve centrifugation or filtration to remove particles and/or insoluble material. The last (e.g. second) step is nanofiltration or ultrafiltration, which serves several purposes:
One purpose is to concentrate the liquid in a cost efficient way to 20-30% solid consistency
Some inhibitors to fermentation will be removed during such filtration,
The taste of the remaining hemicellulose rich solution improves with such filtration, as the taste becomes less bitter

The invention can also be carried out with only one of these steps: i.e. the separation/filtration can be carried out using only the first step (filtration or centrifugation to remove particles and/or insoluble material), only the second step (nano- or ultrafiltration), or both.

After filtration, we have a hemicellulose solution with typically 10-30%, e.g. 20-30% content of solids, mainly hemicellulose. In softwood, the main part of the hemicellulose is oligosaccharides.

Galactoglucomannan is the largest of these in softwood, while it is glucuronoxylan in hardwood.

The hemicellulose from softwood can among other applications be used as feedstock for fermentation and thereafter distillation to ethanol, or as animal feed. Hemicellulose from hardwood is suitable for animal feed, and as feedstock for various products. Hemicellulose from non-wood lignocellulosic materials can among other applications be used as feedstock for fermentation and thereafter distillation to ethanol, or as animal feed. It can also be used as feedstock for various other products.

The properties of the hemicellulose can be compared to molasses, and sugars from wood is sometimes called “wood molasses”.

The liquid fraction (hemicellulose-containing fluid component) may be utilised in any appropriate method including, for example, the generation of biogas (methanisation).

### Optional Evaporation and Drying

A solution with 20-30% hemicellulose may be a commercial product as it is. Optional further processing with evaporation will increase the value due to a higher concentration of the solution. The solution turns into syrup if the percentage of solids are considerably increased through evaporation, as the viscosity increases with the increased percentage of solids.

The hemicellulose solution can be dried to powder using techniques such as spray drying. This form is the most convenient if the product is to be used as animal feed.

### Optional Fermentation and Distillation

Fermentation and distillation is an option for hemicellulose from softwood, but not from hardwood unless additional treatment is undertaken.

Since some inhibitors to fermentation are removed during nano- or ultrafiltration, and there is enough monosaccharides present to start the fermentation process, fermentation can be done directly after the filtration steps (particularly in hemicellulose from softwood). But to ensure a higher yield, one option is to have a hydrolysing step after filtration, a step which comprises heat, acids or enzymes. Such a step would further decrease the level of fermentation inhibitors and/or increase the level of monosaccharaides so as to enhance fermentation.

After fermentation, distillation to ethanol can be done. This ethanol falls within the concept of cellulosic bioethanol, the production of which is a priority in several countries.

### Non-Wood Lignocellulosic Materials—Test Data

Test result for rice straw was as follows:

| | Untreated | Treated | Norm |
|---|---|---|---|
| Ash Deformation | 920° C. | >1500° C. | SIS-CEN/TS15370-1: 2007 |
| Temp, DT (ox.atm.) | | | |

The ‘untreated’ ash deformation temperature corresponds to the ash deformation temperature of the rice straw before processing by the method of the invention. The ‘treated’ ash deformation temperature corresponds to the ash deformation temperature of the solid biomass-derived material obtained after processing

Treatment of other lignocellulosic materials (e.g. bagasse) gives similar increases in ash deformation temperatures.

Another problem with solid fuels from lignocellulosic materials is that in some cases the content of chlorine is too high. Testing of lignocellulosic material showed that our method reduces the chlorine content significantly.

The result for rice straw was as follows:

| | Untreated | Treated | Norm |
|---|---|---|---|
| Chlorine (Cl) dry basis | 0.48% | 0.06% | SS-ENISO16994: 2016 |

Chlorine content of 0.48% is far too high for most combustion equipment, while 0.06% can be acceptable.

The practical value of using the method for non-wood lignocellulosic materials is high. Raw materials such as straw, bagasse and others are available in abundance, and are a potential source of carbon-neutral fuel, alongside wood.

TABLE 1
Analysis of rice straw, before processing

| Analysis | Value | | Norm |
|---|---|---|---|
| Ash, 550° C. db | 19.7% | db | SS-EN ISO 18122: 2015 |
| Carbon (C) db | 39.2% | db | SS-EN ISO 16948: 2015 |
| Hydrogen (H) db | 5.2% | db | SS-EN ISO 16948: 2015 |
| Nitrogen (N) db | 1.52% | db | SS-EN ISO 16948: 2015 |
| Oxygen (O) db | 33.8% | db | Calculated |
| Chlorine (Cl) db | 0.48% | db | SS-EN ISO 16994: 2016 |
| Fluorine (F) db | <0.005#% | db | SS-EN ISO 16994: 2016 |
| Bromine (Br) db | <0.005#% | db | SS-EN ISO 16994: 2016 |
| Sulphur (S) db | 0.125% | db | SS-EN ISO 16994: 2016 |
| Gross cal. value Const volume db | 15.548 | MJ/kg | SS-EN 14918: 2010 |
| Net cal. value Const press db | 14.426 | MJ/kg | SS-EN 14918: 2010 |
| Net cal. value Const press db ashfree | 17.956 | MJ/kg | SS-EN 14918: 2010 |
| Gross cal. value Const volume db | 3713 | Kcal/kg | SS-EN 14918: 2010 |
| Net cal. value Const press db | 3445 | Kcal/kg | SS-EN 14918: 2010 |
| Net cal. value Const press db ashfree | 4288 | Kcal/kg | SS-EN 14918: 2010 |
| Gross cal. value Const volume db | 4.318 | MWh/ton | SS-EN 14918: 2010 |
| Net cal. value Const press db | 4.006 | MWh/ton | SS-EN 14918: 2010 |
| Net cal. value Const press db ashfree | 4.986 | MWh/ton | SS-EN 14918: 2010 |
| Ash Shrinkage Starting Temp, SST (ox.atm.) | 790° | C. | SIS-CEN/TS 15370-1: 2007 |
| Ash Deformation Temp, DT (ox.atm.) | 920° | C. | SIS-CEN/TS 15370-1: 2007 |
| Ash Hemisphere Temp, HT (ox.atm.) | 1380° | C. | SIS-CEN/TS 15370-1: 2007 |
| Ash Flow Temp, FT (ox.atm.) | 1470° | C. | SIS-CEN/TS 15370-1: 2007 |

TABLE 2
Analysis of solid material derived from rice straw after processing

| Analysis | Value | | Norm |
|---|---|---|---|
| Ash, 550° C. db | 19.8% | db | SS-EN ISO 18122: 2015 |
| Carbon (C) db | 42.6% | db | SS-EN ISO 16948: 2015 |
| Hydrogen (H) db | 5.2% | db | SS-EN ISO 16948: 2015 |
| Nitrogen (N) db | 1.27% | db | SS-EN ISO 16948: 2015 |
| Oxygen (O) db | 31.1% | db | Calculated |
| Chlorine (Cl) db | 0.06% | db | SS-EN ISO 16994: 2016 |
| Fluorine (F) db | <0.005#% | db | SS-EN ISO 16994: 2016 |
| Bromine (Br) db | <0.005#% | db | SS-EN ISO 16994: 2016 |
| Sulphur (S) db | 0.073% | db | SS-EN ISO 16994: 2016 |
| Gross cal. value Const volume db | 17.100 | MJ/kg | SS-EN 14918: 2010 |
| Net cal. value Const press db | 15.978 | MJ/kg | SS-EN 14918: 2010 |
| Net cal. value Const press db ashfree | 19.914 | MJ/kg | SS-EN 14918: 2010 |
| Gross cal. value Const volume db | 4083 | Kcal/kg | SS-EN 14918: 2010 |
| Net cal. value Const press db | 3816 | Kcal/kg | SS-EN 14918: 2010 |
| Net cal. value Const press db ashfree | 4756 | Kcal/kg | SS-EN 14918: 2010 |
| Gross cal. value Const volume db | 4.749 | MWh/ton | SS-EN 14918: 2010 |
| Net cal. value Const press db | 4.437 | MWh/ton | SS-EN 14918: 2010 |
| Net cal. value Const press db ashfree | 5.530 | MWh/ton | SS-EN 14918: 2010 |
| Ash Shrinkage Starting Temp, SST (ox.atm.) | 980° | C. | SIS-CEN/TS 15370-1: 2007 |
| Ash Deformation Temp, DT (ox.atm.) | >1500° | C. | SIS-CEN/TS 15370-1: 2007 |
| Ash Hemisphere Temp, HT (ox.atm.) | >1500° | C. | SIS-CEN/TS 15370-1: 2007 |
| Ash Flow Temp, FT (ox.atm.) | >1500° | C. | SIS-CEN/TS 15370-1: 2007 |

The abbreviation ‘db’ in the above tables stands for ‘dry basis’

As can be seen from Tables 1 and 2, the process of the present invention dramatically increases ash defromation temperature and reduces chlorine content.

Various embodiments of the present invention include the following:

1. A method for generating a solid wood-based material and a hemicellulose-derived material from a wood raw material, said method comprising;
i) treating the wood raw material under aqueous conditions at elevated temperature and pressure whereby to generate a hemicellulose-containing fluid component and a solid component;
ii) separating said fluid component from said solid component;
iii) processing at least a part of said solid component into a solid wood-based; and
iv) processing said liquid component into a hemicellulose-derived material.
   2. The method of embodiment 1 wherein the solid wood-based material comprises a fuel, preferably fuel pellets or fuel powder.
   3. The method of embodiment 2 wherein said fuel pellets or fuel powder are wood pellets depleted in hemicellulose.
   4. The method of embodiment 2 or embodiment 3 wherein said fuel pellets have a higher energy density than whole-wood pellets. Similarly, the fuel powder may have an energy density higher than whole-wood powder and/or pellets.
   5. The method of any preceding embodiment wherein said hemicellulose-derived material comprises at least one material selected from; a sugar solution, a syrup, a sugar-containing powder, an aqueous ethanol solution and ethanol.
   6. The method of any preceding embodiment wherein said wood raw material comprises wood chips, wood dust, and/or wood particles.
   7. The method of any preceding embodiment wherein step i) comprises steam explosion of the wood raw material whereby to generate an exploded wood material and optionally washing said exploded wood material with an aqueous material such as water.
   8. The method of embodiment 7 wherein said steam explosion comprises;
a) introducing the wood raw material into a pressure vessel
b) heating the wood raw material by injecting steam and keeping the temperature at 150-280° C. for a period of 60-2400 seconds;
c) reducing the pressure in one or more steps and removing the exploded wood material out of the vessel;
   9. The method of any preceding embodiment wherein step ii) comprises;
d) washing the exploded wood material.
e) separating the exploded wood material and moisture into a solids fraction comprising most (e.g. greater than 90%) of the solids, and a fluids fraction comprising most (e.g. greater than 70%, preferably greater than 80%) of the liquid;
   10. The method of any preceding embodiment wherein step iii) comprises;
f) dewatering and drying the solids fraction to below 20% moisture whereby to generate said solids component.
   11. The method of any preceding embodiment wherein step iv) comprises;
g) filtration of the fluids fraction in at least two steps;
   I) A first filtration step after which the liquid component is retained; and
   II) A second filtration step comprising ultrafiltration or nanofiltration of said liquid component, in which the concentration of hemicellulose in the filtrate is increased;
h) Optionally fermenting the filtrate, followed by distillation to ethanol, or
i) Optionally evaporating the filtrate to a syrup with increased concentration of hemicellulose, and
j) Optionally drying the said syrup to a powder
   12. The method of any preceding embodiment wherein the wood raw material comprises softwood.
   13. The method of any preceding embodiment wherein, wherein the wood raw material comprises hardwood.
   14. The method of any preceding embodiment wherein at step i) the temperature is 180-230° C. or 195-215° C.
   15. The method of embodiment 9 wherein washing is done as counter current washing.
   16. The method of any preceding embodiment wherein at least one enzyme enabling hydrolysis of parts of the cellulose is introduced between steps i) and ii) and followed by an incubation period of up to 36 hours before step ii).
   17. The method of any preceding embodiment wherein at least one enzyme enabling hydrolysis of parts of the cellulose is introduced between steps ii) and iii) and followed by an incubation period of up to 36 hours before step iii).
   18. The method of embodiment 16 or embodiment 17 wherein the hydrolysed cellulose is separated following incubation and optionally processed into a sugar solution, a syrup and/or a sugar-containing powder.
   19. The method of embodiment 9, wherein the solid fraction has a moisture content below 50% on wet basis.
   20. The method of embodiment 10, wherein the solids fraction is dewatered and dried to below 10% moisture on wet basis.
   21. The method of any preceding embodiment wherein in step iii) the solids component is pelletized after adding a carbon rich additive, thereby increasing the fixed C in the pellets.
   22. The method of any preceding embodiment wherein in step iii) the solids component is pelletized after adding an additive rich in fat or oil.
   23. The method of any preceding embodiment wherein in step iii) at least a part of the solids fraction is compressed into a construction material such as beams, boards, or sheets, optionally after adding binding agents.
   24. The method of embodiment 11, wherein the concentration of dissolved material in the filtrate after the last filtration is above 10%.
   25. The method of embodiment 11, wherein the concentration of dissolved material in the filtrate after the last filtration is above 20%.
   26. The method of embodiment 11, wherein the concentration of dissolved material in the filtrate after the last filtration is above 25%.
   27. The process of embodiment 11, wherein the filtrate is hydrolysed by heat, acids or enzymes before the filtrate is optionally fermented.
   28. The process of embodiment 8, wherein reduction of the pressure in the pressure vessel is partly done by injecting water into the pressure vessel.
   29. The process of embodiment 11, wherein drying of the syrup to powder is done in a spray dryer.
   Various other embodiments include
   A. A method for generating a solid wood-based material and a hemicellulose-derived material from a wood raw material, said method comprising;
i) treating the wood raw material under aqueous conditions at elevated temperature and pressure whereby to generate a hemicellulose-containing fluid component and a solid component;
ii) separating said fluid component from said solid component;
iii) processing at least a part of said solid component into a solid wood-based material; and
iv) processing said liquid component into a hemicellulose-derived material.
   B. The method of embodiment A wherein said solid wood-based material comprises a fuel, preferably fuel pellets or fuel powder.
   C. The method of embodiment B wherein said fuel is wood pellets or powder depleted in hemicellulose.
   D. The method of embodiment B or embodiment C wherein said fuel pellets has an higher energy density than whole-wood pellets.
   E. The method as defined in any preceding embodiment wherein said hemicellulose-derived material comprises at least one material selected from; a sugar solution, a syrup, a sugar-containing powder, an aqueous ethanol solution and ethanol.
   F. The method of any preceding embodiment wherein said wood raw material comprises wood chips, wood dust, and/or wood particles.
   G. The method of any preceding embodiment wherein step i) comprises steam treatment, or steam explosion, of the wood raw material whereby to generate a steam treated wood material and optionally washing said treated wood material with an aqueous material such as water.
   H. The method of embodiment G wherein said steam treatment comprises;
a) introducing the wood raw material into a pressure vessel;
b) heating the wood raw material by injecting steam and keeping the temperature at 150-280° C. for a period of 60-2400 seconds;
c) reducing the pressure in one or more steps and removing the exploded wood material out of the vessel.
   I. The method of any preceding embodiment wherein step ii) comprises;
d) washing the exploded wood material.
e) separating the exploded wood material and moisture into a solids fraction comprising most of the solids, and a fluids fraction comprising most of the liquid;
   J. The method of any preceding embodiment wherein step iii) comprises;
f) dewatering and drying the solids fraction to below 20% moisture whereby to generate said solids component.
   K. The method of any preceding embodiment wherein step iv) comprises;
g) filtration of the fluids fraction in at least two steps;
   I) A first separation step removing particles and/or insoluble material, after which the liquid component is retained; and
   II) A second filtration step comprising ultrafiltration or nanofiltration of said liquid component, in which the concentration of hemicellulose in the filtrate is increased;
h) Optionally fermenting the filtrate, followed by distillation to ethanol, or
i) Optionally evaporating the filtrate to a syrup with increased concentration of hemicellulose, and
j) Optionally drying the said syrup to a powder
   L. The process of embodiment K, wherein the filtrate is hydrolysed by heat, acids or enzymes before the filtrate is optionally being fermented.
   M. The process of embodiment K, wherein drying of the syrup to powder is done in a spray dryer.
   N. The process of embodiment H, wherein reduction of the pressure in the pressure vessel is partly done by injecting water into the pressure vessel.
   O. The method of any preceding embodiment wherein the wood raw material comprises softwood and/or hardwood.
   P. The method of any preceding embodiment wherein at step i) the temperature is 150-230° C. or 195-215° C.
   Q. The method of any preceding embodiment wherein at least one enzyme enabling hydrolysis of parts of the cellulose is introduced between steps i) and ii) and followed by an incubation period of up to 36 hours before step ii).
   R. The method of any preceding embodiment wherein at least one enzyme enabling hydrolysis of parts of the cellulose is introduced in the solid component between steps ii) and iii) and followed by an incubation period of up to 36 hours before step iii).
   S. The method of embodiment Q or embodiment R wherein the hydrolysed cellulose is separated following incubation and optionally processed into a sugar solution, a syrup and/or a sugar-containing powder.
   T. The method of embodiment I, wherein the solid fraction has a moisture content below 50% on wet basis.
   U. The method of embodiment J, wherein the solids fraction is dewatered and dried to below 10% moisture on wet basis.
   V. The method of any preceding embodiment wherein in step iii) the solids component is pelletized after adding a carbon rich additive, thereby increasing the fixed C in the pellets.
   X. The method of any preceding embodiment wherein in step iii) the solids component is pelletized after adding an additive rich in fat or oil.
   Y. The method of any preceding embodiment wherein in step iii) at least a part of the solids fraction is compressed into a construction material such as beams, boards, or sheets, optionally after adding binding agents.
   Z. The method of embodiment K, wherein the concentration of dissolved material in the filtrate after the last filtration is above 10%.
   AA. The process of any preceding embodiment wherein the solid wood-based material has an ash content of less than 0.15 wt %.
   BB. The process of any preceding embodiment wherein the solid wood-based material is in the form of particles in which at least 80% by number have a smallest dimension of less than 250 μm.
   CC. The process of any preceding embodiment wherein the solid wood-based material is in the form of particles in which at least 60% by volume have a particle size of less than 250 μm as measured by laser scattering.
   DD. A wood derived fuel having an ash content of less than 0.25 wt %.
   EE. A wood derived fuel of embodiment DD in the form of particles in which at least 80% by number have a smallest dimension of less than 250 μm.
   FF. A wood derived fuel of embodiment DD or embodiment EE in the form of particles in which at least 60% by volume have a particle size of less than 250 μm as measured by laser scattering.
   GG. A wood derived fuel of embodiment DD is in the form of pellets.
   HH. A liquid fuel comprising the wood derived fuel of any of embodiments EE to GG and at least one hydrocarbon liquid.

As recited in the claims, the invention provides the following aspect:

JJ. A method for generating a solid biomass-derived material and a hemicellulose-derived material from a biomass raw material, said method comprising;
i) treating the biomass raw material under aqueous conditions at elevated temperature and pressure whereby to generate a hemicellulose-containing fluid component and a solid component;
ii) separating said fluid component from said solid component;
iii) processing at least a part of said solid component into a solid biomass-based material;
wherein said biomass is a lignocellulosic material;
wherein said solid biomass-derived material has an ash deformation temperature of at least 1000° C.

The invention also provides the following embodiments:

KK. The method of embodiment JJ wherein said solid biomass-based material comprises a fuel, preferably fuel pellets or fuel powder,
LL. The method of embodiment KK preferably wherein said fuel is biomass pellets or powder depleted in hemicellulose.
MM. The method of embodiment KK wherein said fuel pellets has an higher energy density than whole-biomass pellets (i.e. pellets of biomass raw material).
NN. The method of any of embodiments JJ to MM wherein said hemicellulose-derived material comprises at least one material selected from; a sugar solution, a syrup, a sugar-containing powder, an aqueous ethanol solution and ethanol.
OO. The method of any of embodiments JJ to NN wherein said biomass raw material comprises biomass chips, biomass dust, and/or biomass particles
PP. The method of any of embodiments JJ to OO wherein step iii) comprises;
f) dewatering and drying the solids fraction to below 20% moisture whereby to generate said solids component.
   QQ. The method of any of embodiments JJ to PP wherein step iv) comprises;
g) separation of the fluids fraction in at least one of two steps;
   I) A separation step I), e.g. centrifugation or filtration, for removing particles and/or insoluble material, after which the liquid component is retained; and/or
   II) A filtration step II) comprising ultrafiltration or nanofiltration of said liquid component, in which the concentration of hemicellulose in the filtrate is increased;
h) Optionally fermenting the filtrate, followed by distillation to ethanol, or
i) Optionally evaporating the filtrate to a syrup with increased concentration of hemicellulose, and
j) Optionally drying the said syrup to a powder
   RR. The method of embodiment QQ, wherein the filtrate is hydrolysed by heat, acids or enzymes before the filtrate is optionally being fermented.
   SS. The method of embodiment QQ, wherein drying of the syrup to powder is done in a spray dryer.
   TT. The method of any of embodiments JJ to SS, wherein step i) comprises steam treatment, or steam explosion, of the biomass raw material whereby to generate a steam treated biomass material and optionally washing said treated biomass material with an aqueous material such as water, wherein said steam treatment comprises;
a) introducing the biomass raw material into a pressure vessel;
b) heating the biomass raw material by injecting steam and keeping the temperature at 150-280° C. for a period of 60-2400 seconds;
c) reducing the pressure in one or more steps and removing the exploded biomass material out of the vessel;
   and wherein reduction of the pressure in the pressure vessel is partly done by injecting water into the pressure vessel.
   UU. The method of any of embodiments JJ to TT wherein at least one enzyme enabling hydrolysis of parts of the cellulose is introduced between steps i) and ii) and followed by an incubation period of up to 36 hours before step ii).
   VV. The method of any of embodiments JJ to UU wherein at least one enzyme enabling hydrolysis of parts of the cellulose is introduced in the solid component between steps ii) and iii) and followed by an incubation period of up to 36 hours before step iii).
   XX. The method of embodiment UU or VV wherein the hydrolysed cellulose is separated following incubation and optionally processed into a sugar solution, a syrup and/or a sugar-containing powder.
   ZZ. The method of any of embodiments JJ to XX, wherein step ii) comprises;
d) washing the exploded biomass material.
e) separating the exploded biomass material and moisture into a solids fraction comprising most of the solids, and a fluids fraction comprising most of the liquid;
   and wherein the solid fraction has a moisture content below 50% on wet basis.
   AAA. The method of embodiment PP, wherein the solids fraction is dewatered and dried to below 10% moisture on wet basis.
   BBB. The method of any of embodiments JJ to AAA wherein in step iii) the solids component is pelletized after adding a carbon rich additive, thereby increasing the fixed C in the pellets.
   CCC. The method of any of embodiments JJ to BBB wherein in step iii) the solids component is pelletized after adding an additive rich in fat or oil.
   DDD. The method of any of embodiments JJ to CCC wherein in step iii) at least a part of the solids fraction is compressed into a construction material such as beams, boards, or sheets, optionally after adding binding agents.
   EEE. The method of embodiment QQ, wherein the concentration of dissolved material in the filtrate after the last filtration is above 10%, e.g. above 20% or above 25%.
   FFF. The method of any of embodiments JJ to EEE wherein the solid biomass-based material is in the form of particles in which at least 80% by number have a smallest dimension of less than 250 μm.
   GGG. The method of any of embodiments JJ to FFF wherein the solid biomass-based material is in the form of particles in which at least 60% by volume have a particle size of less than 250 μm as measured by laser scattering.
   HHH. The method of embodiment JJ, comprising the steps of:
i) steam treating, or steam exploding, the biomass raw material to generate a hemicellulose-containing fluid component and a solid biomass-based component, wherein said steam treating or steam exploding method comprising the steps of includes:
   a) introducing the biomass raw material into a pressure vessel;
   b) heating the biomass raw material by injecting steam and keeping the temperature at 195-230° C. for a period of 60-1200 seconds;
   c) reducing the pressure in one or more steps and
   d) removing the exploded biomass material out of the vessel;
ii) separating said hemicellulose-containing fluid component from said solid biomass-based component;
iii) processing at least a part of said solid biomass-based component into a solid biomass-based material; and
iv) processing said hemicellulose-containing fluid component into a hemicellulose-derived material by separating (e.g. filtering) the fluids fraction in at least one (e.g. at least two steps), wherein the filtering includes:
   I) removing particles and/or insoluble material, e.g. by centrifugation or filtration, after which the liquid component is retained; and/or
   II) ultrafiltration or nanofiltration of said hemicellulose-containing fluid component, in which the concentration of hemicellulose in the filtrate is increased; and
   wherein the concentration of dissolved material in the filtrate after the last filtration is above 10%;
wherein said biomass is a non-wood lignocellulosic material; and
wherein said solid biomass-derived material has an ash deformation temperature of at least 1000° C.

What is claimed:

1. A method for generating a solid biomass-derived material and a hemicellulose-derived material from a biomass raw material, said method comprising;
i) treating the biomass raw material under aqueous conditions at elevated temperature and pressure whereby to generate a hemicellulose-containing fluid component and a solid component;
ii) separating said fluid component from said solid component;
iii) processing at least a part of said solid component into a solid biomass-based material;
wherein said biomass is a lignocellulosic material;
wherein said solid biomass-derived material has an ash deformation temperature of at least 1000° C.

2. A method as claimed in claim 1, further comprising a step of iv) processing said liquid component into a hemicellulose-derived material.

3. A method as claimed in claim 1, wherein said biomass is a non-wood lignocellulosic material, such as straw, bagasse, stover, grass or any mixtures thereof, preferably straw, bagasse, or any mixtures thereof.

4. A method as claimed in claim 1, wherein said solid biomass-derived material has an ash deformation temperature of at least 1050° C., preferably at least 1100° C., preferably at least 1200° C., more preferably at least 1300° C.

5. A method as claimed in claim 1, wherein said solid biomass-derived material has a chlorine content of 0.2 wt % or less, preferably 0.1 wt % or less, more preferably 0.08 wt % or less.

6. A method as claimed in claim 1, wherein the process is carried out in the absence of additives for increasing the ash melting temperature, such as mineral agents, e.g. calcium carbonate, lime or limestone.

7. The method of claim 1 wherein step i) comprises steam treatment, or steam explosion, of the biomass raw material whereby to generate a steam treated biomass material and optionally washing said treated biomass material with an aqueous material such as water.

8. The method of claim 7 wherein said steam treatment comprises;
a) introducing the biomass raw material into a pressure vessel;
b) heating the biomass raw material by injecting steam and keeping the temperature at 150-280° C. for a period of 60-2400 seconds;
c) reducing the pressure in one or more steps and removing the exploded biomass material out of the vessel.

9. The method of claim 1 wherein step ii) comprises;
d) washing the exploded biomass material.
e) separating the exploded biomass material and moisture into a solids fraction comprising most of the solids, and a fluids fraction comprising most of the liquid;

10. The method of claim 1 wherein step iv) comprises;
g) separation of the fluids fraction in at least one of two steps;
I) A separation step I), e.g. centrifugation or filtration, for removing particles and/or insoluble material, after which the liquid component is retained; and/or
II) A filtration step II) comprising ultrafiltration or nanofiltration of said liquid component, in which the concentration of hemicellulose in the filtrate is increased;
h) Optionally fermenting the filtrate, followed by distillation to ethanol, or
i) Optionally evaporating the filtrate to a syrup with increased concentration of hemicellulose, and
j) Optionally drying the said syrup to a powder.

11. The method of claim 1 wherein at step i) the temperature is 150-230° C. or 195-215° C.

12. The method of claim 1 wherein the solid biomass-based material has an ash content of less than 0.15 wt %.

13. The method of claim 1, comprising the steps of:
i) steam treating, or steam exploding, the biomass raw material to generate a hemicellulose-containing fluid component and a solid biomass-based component, wherein said steam treating or steam exploding method comprising the steps of includes:
a) introducing the biomass raw material into a pressure vessel;
b) heating the biomass raw material by injecting steam and keeping the temperature at 195-230° C. for a period of 60-1200 seconds;
c) reducing the pressure in one or more steps; and
d) removing the exploded biomass material out of the vessel;
ii) separating said hemicellulose-containing fluid component from said solid biomass-based component;
iii) processing at least a part of said solid biomass-based component into a solid biomass-based material; and
wherein said biomass is a non-wood lignocellulosic material; and
wherein said solid biomass-derived material has an ash deformation temperature of at least 1000° C.

14. A solid biomass-derived material having
an ash deformation temperature of at least 1000° C.
an ash content of less than 0.25 wt %,
wherein said biomass is a non-wood lignocellulosic material.

15. A solid biomass-derived material as claimed in claim 14,
wherein said biomass is selected from straw, bagasse, stover, grass or any mixtures thereof.

16. A solid biomass-derived material as claimed in claim 14, having an ash deformation temperature of at least 1050° C., preferably at least 1100° C., preferably at least 1200° C., more preferably at least 1300° C.

17. A solid biomass-derived material as claimed in claim 14, having a chlorine content of 0.2 wt % or less, preferably 0.1 wt % or less, more preferably 0.08 wt % or less, more preferably 0.07 wt % or less.

18. A solid biomass-derived material of claim 14 in the form of particles in which at least 80% by number have a smallest dimension of less than 250 μm; or in the form of particles in which at least 60% by volume have a particle size of less than 250 μm as measured by laser scattering.

19. A solid biomass-derived material of claim 14 in the form of pellets.

20. A liquid fuel comprising the solid biomass-derived material of claim 14 and at least one hydrocarbon liquid.

21. A solid biomass-derived material obtained or obtainable by the method of claim 1.
